(19) United States
(12) Patent Application Publication
(10) Pub. No.: US 2008/0053462 A1
(43) Pub. Date: Mar. 6, 2008

(54) Surgical drape with convective heat therapy device

(76) Inventors: Leonides Y. Teves, Bradenton, FL (US); Steven D. McCarus, Orlando, FL (US); Scott Gammons, Loudon, TN (US)

Correspondence Address: KNOX PATENTS, P.O. BOX 30034, KNOXVILLE, TN, 37930-0034, US

(21) Appl. No.: 11/469,947

(22) Filed: Sep. 5, 2006

(51) Int. Cl. A61B 19/00; A61F 5/37

(52) U.S. Cl. 128/849

(57) ABSTRACT

An apparatus for a heated surgical drape device including a surgical drape with an integral convective heat therapy portion. The surgical drape is attached to the convective heat therapy portion. In various embodiments, the surgical drape includes a surgical drape with removable sections that allow access to the body of the patient during surgery, at least one pocket, and an integral window adjacent the head of the patient to allow viewing of the head of the patient. The convective heat therapy portion is a tubular device formed of sheets joined together and sized to allow the patient to rest within a pair of tube sections. The convective heat therapy portion has a plurality of exhaust openings that exhaust heated air toward the patient. In one embodiment, the tubular device has wing sections that are adjacent the outstretched arms of the patient.

### CROSS-REFERENCE TO RELATED APPLICATIONS

Not Applicable

### STATEMENT REGARDING FEDERALLY SPONSORED RESEARCH OR DEVELOPMENT

Not Applicable

### BACKGROUND OF THE INVENTION

1. Field of Invention

This invention relates to medical appliances. More particularly, it relates to a surgical drape with an integrated convective heat therapy device for avoiding hypothermic conditions in a patient undergoing surgery.

2. Description of the Related Art

Medical surgical procedures are typically performed in operating rooms where the temperature is maintained at about 63 degrees Fahrenheit. Although the medical staff can dress appropriately for the cool temperature, the patient is usually in a state of undress and can experience hypothermia if not protected from the cold temperature. Although the patient may be covered with a surgical drape, such a drape is not intended to and do not function to keep the patient warm.

It is known to apply heat to a patient in order to replace the heat that is lost and to prevent hypothermia. One such device is disclosed in U.S. Pat. No. 6,820,622, issued to Teves, et al., on Nov. 23, 2004, titled “Thermal surgical drape.” The Teves patent discloses a surgical drape and warming blanket that “covers the entire body of a supine patient to provide draping and body temperature management in a single structure.” The disclosed combination includes a substrate of a flexible material for placing over a patient and a flexible sheet that overlies and is sealed to the substrate. Warm compressed air is introduced to the space between the substrate and the flexible sheet. The flexible sheet has vent holes to allow the compressed air to flow through the device and out away from the patient. The device includes a removably mounted panel allowing access to the patient and an attached platform for supporting tools used by the medial staff.

One method for controlling a temperature of a patient is disclosed in U.S. Pat. No. 6,167,885, issued Hanssen on Jan. 2, 2001, titled “Method and apparatus for controlling the body temperature of a patient.” This patent discloses a surgical drape 3 used in combination with a warm-air unit 9 that blows warmed air through three outlet pipes 11, 12, 13 that are disposed under the drape 3. The outlet pipes 11, 12, 13 are adjacent the lower extremities of the patient 1.

Another surgical drape is disclosed in U.S. Pat. No. 5,800,483, issued to Vought on Sep. 1, 1998, and titled “System and method for sterile surgical-thermal drape with active air circulation.” The Vought patent discloses a surgical drape 10 made to cover a patient and that includes a flexible drape 12 and a thermal device 14. The thermal device 14 is either an active or a passive device. One disclosed embodiment of the drape 110 includes passageways 116 formed between the drape layer 112 and the thermal surface 114.

Another surgical drape is disclosed in U.S. Pat. No. 5,522,871, issued to Sternlicht on Jun. 4, 1996, and titled “Apparatus for controlling the body temperature of a patient.” This patent disclose an apparatus 33 with a flexible corrugated air hose 14 comprising an air hose assembly 33b that, in one embodiment illustrated in FIG. 7, is loosely attached to the underside of a surgical drape 33a with a plurality of flexible attachment members 33c. The air hose 14 is not disclosed as being an inflatable device.

A convective heat therapy device is disclosed in U.S. Pat. No. 6,827,729, issued to Gammons, et al., on Dec. 7, 2004, titled “Convective heat therapy device.” The Gammons patent discloses a convective heat therapy device 1410 with tubes 1402, 1404 that extend alongside the body of a patient 110. One embodiment of the device 10 for providing heat therapy to the head portion of the patient 110 includes a base sheet 104 upon which the patient 100 rests and a hingedly attached cover sheet 106 disposed over the face of the patient. Another embodiment of the heat therapy device 1410 includes two tubular legs 1402, 1404 with a connecting crosspiece 1406. Although the patent discloses that a sheet or blanket can be placed over the therapy device 1410 to keep the exhausted air from escaping the area of the body, the patent does not disclose a means of attaching the blanket to the device 1410. Neither does the patent disclose using a surgical drape in conjunction with the device 1410, with such a drape having access to the patient such as physical access through a removable panel or visual access through a window in the drape.

### BRIEF SUMMARY OF THE INVENTION

According to one embodiment of the present invention, a heated surgical drape device is provided. The heated surgical drape device includes a surgical drape with an integral convective heat therapy portion. The surgical drape is attached to the therapy portion to contain the exhausted air that warms the patient. Also, the surgical drape is attached to the therapy device to provide stability to the surgical drape when the device is initially positioned over the patient and when the convective therapy portion is inflated. In one embodiment, the surgical drape is attached to the convective heat therapy portion with double-sided tape. In another embodiment, the attachment of the surgical drape to the convective heat therapy portion is with an adhesive or a flexible welded or heat sealed connection.

In one embodiment, the surgical drape includes a surgical drape with a removable section, or access panel, that allows access to the body of the patient during surgery. In various embodiments the removable section is perforated or partially pre-cut to allow easy removal after the heated surgical drape device is positioned over the patient. The surgical drape is suited for laparoscopic surgery with the surgeon gaining access to the patient through the removable access panel.

In another embodiment, the surgical drape includes an integral window adjacent the head of the patient to allow viewing of the area surrounding the head of the patient. With the head of the patient visible, the medical staff is better able to avoid making inadvertent contact with the head, which could injure the patient or dislodge an appliance connected to or near the patient.

The convective heat therapy portion is a tubular device sized to allow the body portion of the patient to rest within a pair of tube sections. In one embodiment, the tubular device has a pair of tubes that extend alongside substantially the full length of the patient, that is, from head to foot. A crosspiece connects the pair of tubes and includes an inlet port for receiving the heated air. In another embodiment, each tube of the pair of tubes of the tubular device has a wing section that is adjacent one of the outstretched arms of the patient. Such an embodiment accommodates a patient that must be operated upon with the arms in the outstretched position. The convective heat therapy portion has a plurality of exhaust openings that exhaust heated air toward the patient.

### BRIEF DESCRIPTION OF THE SEVERAL VIEWS OF THE DRAWINGS

The above-mentioned features of the invention will become more clearly understood from the following detailed description of the invention read together with the drawings in which:

FIG. 1 is a top view of one embodiment of a surgical drape with an integral convective therapy device;

FIG. 2 is a perspective view of one embodiment of the separate components of the surgical drape with an integral convective therapy device;

FIG. 3 is a top view of another embodiment of a surgical drape with an integral convective therapy device.

### DETAILED DESCRIPTION OF THE INVENTION

An apparatus for a heated surgical drape device is disclosed. A feature of the present invention is that the apparatus is made of thin sheets of material and is foldable into a compact space.

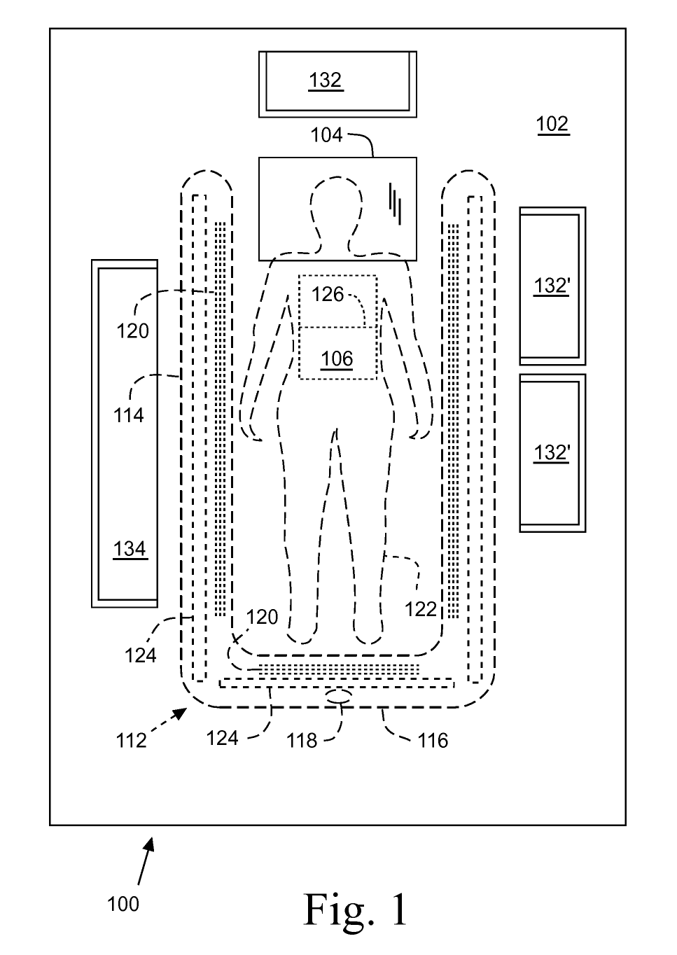

FIG. 1 illustrates a top view of one embodiment of a heated surgical drape device 100 that includes a surgical drape 102 with an integral convective therapy device 112. The drape 102 covers a patient 122 who lies within the convective therapy device 112. When covering a patient 122 during surgery, the drape 102 is typically placed on top of the patient 122 and the portion of the drape 102 that would cover the head of the patient 122 is lifted and suspended to enable the anesthesiologist to access the head of the patient 122.

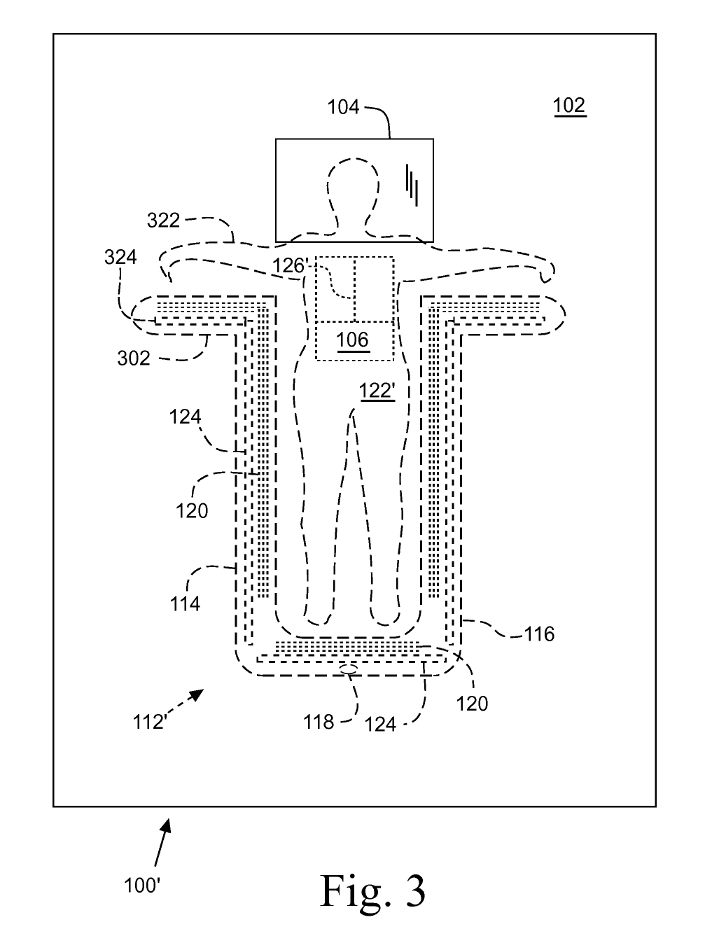

A surgical drape 100 includes an access panel 106 that allows access to the patient 122 from above the surgical drape 102. In the illustrated embodiment, the access panel 106 is a rectangular section that has a perforated boundary. The perforated boundary allows for the separation of the access panel 106 from the remainder of the drape 102. In one embodiment, the access panel 106 includes a dividing perforation 126 that allows flexibility in the size and location of the opening through the surgical drape 102. In the illustrated embodiment, the center dividing perforation 126 allows either the top portion of the access panel 106, the bottom portion of the panel 106, or the complete access panel 106 to be removed from the surgical drape 102. Those skilled in the art will recognize that the location, size, and number of the access panels 106 and dividing perforations 126 can be varied based upon the type of surgery being performed and the needs of the medical staff without departing from the spirit and scope of the present invention. FIG. 3 illustrates another embodiment of the dividing perforations 126′ for the access panel 106 in the surgical drape 102. This embodiment of the dividing perforations 126′ provides for the access panel 106 to be removed in three independent sections.

The surgical drape 102 includes a viewing panel 104 that is a window in the surgical drape 102 that allows viewing of the area surrounding the head of the patient 122. In a typical operating room environment, the patient 122 is covered with an opaque surgical drape. Modern operating rooms and medical practice involve the use of many appliances and medical devices, including aspirators, tubes, and other specialized equipment, often mounted on articulated arms or stands. An opaque drape does not allow viewing of the patient 122; therefore, the exact location of the patient's extremities are not easily determined during operating room procedures. It has been known for operating room personnel to inadvertently strike the patient when moving and/or repositioning appliances and equipment. Striking the patient 122 on or near the head often dislodges aspirators and/or other equipment or injures the patient 122. Accordingly, the placement of the viewing panel 104 allows the medical staff to readily determine the location of the patient's head and the location of appliances and/or equipment connected to the head of the patient 122. In one embodiment, the viewing panel 104 is a clear sheet of flexible material. In another embodiment, the viewing panel 104 is a sheet of flexible, translucent material. In still another embodiment, the viewing panel 104 is a rigid panel.

Below the surgical drape 102 and illustrated with hidden lines is a convection heat therapy portion 112 of the heated surgical drape device 100. The convection heat therapy portion 112 is a U-shaped tube 112 that includes a pair of tubular legs 114 connected at one end with a tubular crosspiece 116. The crosspiece 116 includes an inlet opening, or port, 118 to which a heated air supply tube is connected to inflate the convection heat therapy portion 112. Those skilled in the art will recognize that the inlet opening 118 can be located at a convenient location on the convection heat therapy portion 112 without departing from the spirit and scope of the present invention.

Along the inside of the U-shaped portion of the convection heat therapy portion 112 are a plurality of exhaust ports 120 positioned to exhaust the heated air that is used to inflate the convection heat therapy portion 112. In various embodiments the exhaust ports 120 are round holes, slits, V-shaped beaks, and/or a section of air permeable material. The number and position of the exhaust ports 120 are such that the heated air that is introduced into the inlet port 118 is sufficient to inflate the convection heat therapy portion 112 and to provide a supply of heated air directed toward selected areas of the body of the patient 122. In the illustrated embodiment, the exhaust ports 120 are positioned along the inside of each leg 114 and the crosspiece 116.

The convection heat therapy portion 112 is attached to the surgical drape 102 such that the heated surgical drape device 100 is a single, integral device. In the illustrated embodiment, lengths of double-sided tape 124 are disposed between the convection heat therapy portion 112 and the surgical drape 102. The tape 124 connects the surgical drape 102 to the convection heat therapy portion 112 with a flexible bond that allows the heated surgical drape device 100 to be readily positioned over the patient 122 and to maintain stability of the surgical drape 102 over the patient 122 when the convection heat therapy portion 112 is inflated with air. In another embodiment, the convection heat therapy portion 112 is attached to the surgical drape 102 by welding or heat sealing the surgical drape 102 to the convection heat therapy portion 112. In still another embodiment, the convection heat therapy portion 112 is attached to the surgical drape 102 with an adhesive that bonds the two parts 112, 102 together with a flexible connection.

In the illustrated embodiment, various pockets 132, 132′, 134 are positioned around the periphery of the drape 102. The pockets 132, 132′, 134 allow the surgeon and other medical staff to temporarily store appliances and other tool and devices when not being immediately used. A top pocket 132 is positioned on the drape 102 above the head of the patient 122. This pocket 132 has an opening facing the nearest edge of the drape 102 such that the pocket is operable when the top edge of the drape 102 is suspended to expose the head of the patient 122. Alongside the patient 122 are other pockets 132′, 134. On one side is a long pocket 134 that extends a substantial length of the patient's body. The long pocket 134 has its opening facing the patient 122 because, when covering the patient 122, the drape 102 hangs over the edge of the operating table and the long pocket 134 is vertically oriented with the opening facing upwards. On the opposite side of the patient 122 are a pair of short pockets 132′. Those skilled in the art will recognize that the number, location, and size of the pockets 132, 132′, 134 will vary depending upon the surgical procedure being performed.

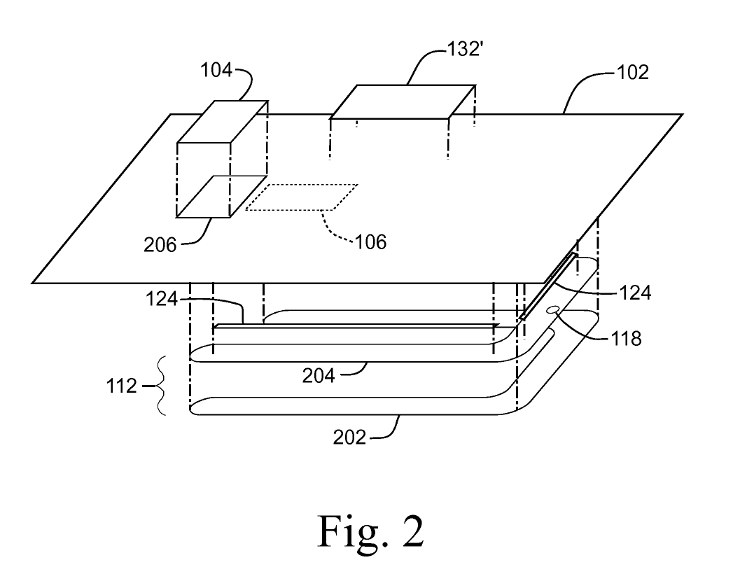

FIG. 2 illustrates a perspective view of one embodiment of the separate components of the surgical drape device 100. The convection heat therapy portion 112 includes a bottom, or lower, sheet 202 and an upper sheet 204 of similar size and configuration to form the U-shaped tube 112. In one embodiment, the sheets 202, 204 are air impermeable, for example, a non-woven fiber ply with a plastic backing. The two sheets 202, 204 are attached at a peripheral edge to form an air impermeable joint such that, when inflated, the two sheets 202, 204 assume a tubular shape. In the illustrated embodiment, the inlet port 118 is formed in the upper sheet 202, although one skilled in the art will recognize that the inlet port 118 can instead be located in the lower sheet 202. In various embodiments, the exhaust ports 120 are formed in one or both of the sheets 202, 204.

The illustrated surgical drape 102 includes the access panel 106 and an opening 206 for receiving the viewing panel 104. In one embodiment, the viewing panel 104 is sized larger than the opening 206 and the overlapping edges of the viewing panel 106 are attached to surgical drape 102. In various embodiments, the viewing panel 104 is bonded to the surgical drape 102 with an adhesive, a double-sided tape, or a welded or heat sealed connection.

A single short pocket 132′ is illustrated in FIG. 2. The pocket 132′ is a rectangular section of material that is bonded to the drape 102 in the desired locations and with the desired orientation. In various embodiments, the pocket 132′ is bonded to the drape 102 with an adhesive, a double-sided tape, or a welded or heat sealed connection. In another embodiment, the pocket 132′ is releasably bonded to the drape 102 with a hook-and-loop fastening system or with snap fasteners.

In the illustrated embodiment, between the surgical drape 102 and the upper sheet 204 are strips of double-sided tape 124. The tape 124 secures the surgical drape 102 to the upper sheet 204. In other embodiments, as discussed elsewhere, an adhesive or a welded connection bonds the surgical drape 102 to the upper sheet 204.

One method of fabricating the heated surgical drape device 100 is to first cut and shape the lower and upper sheets 202, 204 and then join them at their peripheral edges. The exhaust ports 20, in various embodiments, are formed in one or both sheets 202, 204 before or after the sheets 202, 204 are joined together. The opening 206 for the viewing panel 104 is cut in the surgical drape 102 and the viewing panel 104 is attached to the surgical drape 102. The perforations, including the dividing perforations 126, for the access panel 106 are cut into the surgical drape 102. Then the surgical drape 102 is attached to the upper sheet 204. The completed heated surgical drape device 100 is then folded such that it can be readily unfolded to cover the patient. Because the surgical drape 102 must be sterile to be used in an operating room, in one embodiment, the fabrication of the heated surgical drape device 100 is conducted in a clean room environment with sterile materials.

FIG. 3 illustrates top view of another embodiment of a heated surgical drape device 100′ that includes a surgical drape 102 with an integral convective therapy device 112′. In the illustrated embodiment, the patient 122′ has outstretched arms 322. For various medical procedures, this posture is desirable. Also, during many surgical procedures a pulse oximeter is used to monitor the condition of the patient 122′, and it is common to attach the pulse oximeter to a finger of the patient 122′. In a cold operating room, if the fingers of the patient 122′ are not kept warm, blood vessels may constrict, which affects the operation of the pulse oximeter. In the illustrated embodiment, the heated surgical drape device 100′ includes a pair of extensions, or wings, 302 that extend from the ends of the legs 114 opposite the end connected to the crosspiece 116. Each extension 302 is sufficiently long to provide exhaust air to the arm 322 of the patient 122′.

In the illustrated embodiment, additional sections of double-sided tape 324 are disposed between the surgical drape 102 and the convection heat therapy portion 112′. In other embodiments, the surgical drape 102 is attached to convection heat therapy portion 112′ as described previously. An advantage to attaching the extensions 302 to the surgical drape 102 is that when the heated surgical drape device 100′ is initially positioned over a patient 122′, the extensions 302 are disposed at the proper location and with the proper orientation.

The heated surgical drape device 100, 100′ includes various functions. The function of providing a sterile barrier over the patient 122, 122′ is implemented, in one embodiment, by the surgical drape 102. The function of warming the patient 122, 122′ is implemented, in one embodiment, by the convection heat therapy portion 112, 112′, which includes a pair of sheets 202, 204 joined at a peripheral edge.

The function of supplying heated air to the patient 122, 122′ is implemented, in one embodiment, by the convection heat therapy portion 112, 112′. The function of warming the outstretched arms 322 of a patient 122′ is implemented, in one embodiment, by the extensions, or wings, 302 of the convection heat therapy portion 112′, as illustrated in FIG. 3.

The function of connecting the surgical drape 102 to the convection heat therapy portion 112, 112′ is implemented, in one embodiment, by double-sided tape 124 between a sheet 204 of the convection heat therapy portion 112, 112′ and the surgical drape 102. In other embodiments, the function is implemented by an adhesive disposed between the sheet 204 and the surgical drape 102 or by welding or heat sealing the sheet 204 to the surgical drape 102.

The function of providing access to the patient 122, 122′ is implemented, in one embodiment, by the access panel 306. The function of providing a viewing port of the area surrounding the head of the patient 122, 122′ is implemented, in one embodiment, by the viewing panel 104.

From the foregoing description, it will be recognized by those skilled in the art that a heated surgical drape device 100 has been provided. In one embodiment, the device 100 includes a surgical drape 102 attached to a tubular member 112 and the tubular member 112 is sized to receive the body of a patient 122. In another embodiment, the device 100′ includes a surgical drape 102 attached to a tubular member 112′ with tubular extensions 302 and the tubular member 112′ is sized to receive the body of a patient 122′ with the tubular extensions 302 positioned to be adjacent the outstretched arms 322 of the patient 122′. In both embodiments the tubular member 112, 112′ includes a plurality of exhaust ports 120 for discharging heated air toward a patient 122, 122′.

Because the heated surgical drape device 100, 100′ is fabricated of material that is thin sheets 102, 104, 202, 204, 124, the heated surgical drape device 100, 100′ is foldable into a compact package for shipping and for disposal after being used. Further, the heated surgical drape device 100, 100′ is easily placed in service by positioning the folded device 100, 100′ on the patient and unfolding the device 100, 100′ such that the surgical drape 102 is over the patient 122, 122′ and the tubular member 112, 112′ is adjacent the sides of the patient 122, 122′. The integral device 100, 100′ ensures proper placement of the tubular member 112, 112′ relative to the patient 122, 122′ with little or minimal interaction required by medical staff.

Additionally, disposal of the heated surgical drape device 100, 100′ after being used is facilitated by the thin sheets 102, 104, 202, 204, 124 requiring a small volume. Further, the materials used are compatible with incineration disposal methods.

While the present invention has been illustrated by description of several embodiments and while the illustrative embodiments have been described in considerable detail, it is not the intention of the applicant to restrict or in any way limit the scope of the appended claims to such detail. Additional advantages and modifications will readily appear to those skilled in the art. The invention in its broader aspects is therefore not limited to the specific details, representative apparatus and methods, and illustrative examples shown and described. Accordingly, departures may be made from such details without departing from the spirit or scope of applicant's general inventive concept.

What is claimed is:

1. An apparatus for providing a sterile covering and for warming a patient during surgery, said apparatus comprising:
a drape having a length and a width sufficient to cover the patient;
a viewing window disposed in said drape, said viewing window allowing observation of an area around a head of the patient when said drape is positioned adjacent the patient;
an access panel disposed in said drape, said access panel removable from said drape; and
a convection heat therapy device having a pair of leg members and a cross-member connecting said pair of leg members, said convection heat therapy device including a pair of sheets joined at a peripheral edge, said pair of leg members and said cross-member having a U-shape sized to receive the patient between said pair of leg members, said convection heat therapy device having an inlet port for receiving conditioned air, said convection heat therapy device having a plurality of exhaust ports, said plurality of exhaust ports positioned to exhaust air to a volume defined between said pair of legs, and said pair of legs attached to said drape.

2. The apparatus of claim 1 wherein said convection heat therapy device includes a pair of extensions each connected to a corresponding one of said pair of leg members at an end opposite said cross-member, each one of said pair of extensions extending away from an other one of said pair of extensions, said pair of extensions positioned to be adjacent a pair of outstretched arms of the patient when the patient is received by said U-shape between said pair of leg members, said plurality of exhaust ports extending along said pair of extensions and directed toward said pair of outstretched arms of the patient.

3. The apparatus of claim 1 further including at least one pocket disposed on a surface of said drape, said surface of said drape being opposite the patient when said drape is adjacent the patient.

4. The apparatus of claim 1 further including means for warming a pair of outstretched arms of the patient.

5. The apparatus of claim 1 wherein said access panel is connected to said drape with a perforated boundary.

6. The apparatus of claim 1 further including means for connecting said drape to said convection heat therapy device.

7. The apparatus of claim 1 wherein said pair of legs are attached to said drape with one of a double-sided tape, an adhesive, and a welded connection.

8. An apparatus for providing a sterile covering and for warming a patient during surgery, said apparatus comprising:
a means for providing a sterile barrier over the patient;
a means for warming the patient; and
a means for connecting said means for providing said sterile barrier and said means for warming the patient;
whereby said means for providing said sterile barrier and said means for warming the patient are integrated into one unit.

9. The apparatus of claim 8 further including a means for providing access to the patient through said means for providing said sterile barrier.

10. The apparatus of claim 8 further including a means for providing a viewing port of an area around a head of the patient through said means for providing said sterile barrier.

11. The apparatus of claim 8 wherein said means for warming the patient includes a tubular member having a U-shape, said tubular member including a pair of sheets joined at a peripheral edge, said tubular member having a plurality of exhaust ports positioned to direct exhausted air toward a center of said U-shape.

12. The apparatus of claim 8 wherein said means for warming the patient includes a tubular member having a U-shape with a pair of wings extending from an open end of said U-shape, said tubular member including a pair of sheets joined at a peripheral edge, said extending wings adapted to be adjacent a patient received by said U-shape, said tubular member having a plurality of exhaust ports positioned to direct exhausted air toward the patient when received by said tubular member.

13. The apparatus of claim 8 wherein means for connecting includes a connection of said means for providing said sterile barrier to said means for warming the patient formed with one of a double-sided tape, an adhesive, and a welded connection.

14. An apparatus for providing a sterile covering and for warming a patient during surgery, said apparatus comprising:
a drape having a length and a width sufficient to cover the patient;
a tubular member having a U-shape, said tubular member including a pair of sheets joined at a peripheral edge, said tubular member having an inlet port for receiving warmed air, said tubular member having a plurality of exhaust ports positioned to direct exhausted air toward a center of said U-shape; and
a means for connecting said drape to said tubular member;
whereby said drape is held in a fixed position relative to said tubular member when said tubular member is receiving said warmed air.

15. The apparatus of claim 14 further including a viewing window disposed in said drape, said viewing window allowing observation of an area around a head of the patient when said drape is positioned adjacent the patient.

16. The apparatus of claim 14 further including an access panel disposed in said drape, said access panel removable from said drape at a perforated boundary.

17. The apparatus of claim 14 further including means for warming a pair of outstretched arms of the patient.

18. The apparatus of claim 14 wherein said tubular member includes a pair of wings extending from an opening of said U-shape, each one of said pair of wings extending away from an other one of said pair of wings, said pair of wings positioned to be adjacent a pair of outstretched arms of the patient when the patient is received by said U-shape, said plurality of exhaust ports extending along said pair of wings and directed toward said pair of outstretched arms of the patient.

19. The apparatus of claim 14 wherein means for connecting includes a connection of said drape to said tubular member formed with one of a double-sided tape, an adhesive, and a welded connection.

20. The apparatus of claim 14 further including at least one pocket disposed on a surface of said drape, said surface of said drape being opposite the patient when said drape is adjacent the patient.
